(19) United States
(12) Patent Application Publication
(10) Pub. No.: US 2018/0279858 A1
(43) Pub. Date: Oct. 4, 2018

(54) TREATMENT INSTRUMENT INSERTION TOOL

(71) Applicant: OLYMPUS CORPORATION, Tokyo (JP)

(72) Inventors: Fuminori WAKE, Tokyo (JP); Yuki HOSHINO, Sagamihara-shi (JP); Seiko HAMADA, Tokyo (JP)

(73) Assignee: OLYMPUS CORPORATION, Tokyo (JP)

(21) Appl. No.: 16/002,286

(22) Filed: Jun. 7, 2018

Related U.S. Application Data
(63) Continuation of application No. PCT/JP2016/082267, filed on Oct. 31, 2016.

(30) Foreign Application Priority Data
Dec. 18, 2015 (JP) 2015-247698

(51) Int. Cl. A61B 1/00; A61B 1/018; A61B 17/00

(52) U.S. Cl. CPC A61B 1/00133; A61B 1/00137; A61B 2017/0034; A61B 1/018; A61B 17/00234; A61B 1/0014

(57) ABSTRACT

A treatment instrument insertion tool includes a tubular treatment instrument guide portion, configured to guide a treatment instrument into an insertion portion of an insertion device via a treatment instrument insertion port, a guide portion body freely attachably/detachably provided in a region of the insertion portion located outside a subject when the insertion portion is inserted into the subject, and a film member including a hole that allows insertion of the treatment instrument, water-tightly engaging with the treatment instrument when the treatment instrument is inserted into the hole, and being formed of a material freely expandable/contractible and restricting permeation of a liquid.

### CROSS REFERENCE TO RELATED APPLICATION

This application is a continuation application of PCT/JP2016/082267 filed on Oct. 31, 2016 and claims benefit of Japanese Application No. 2015-247698 filed in Japan on Dec. 18, 2015, the entire contents of which are incorporated herein by this reference.

### BACKGROUND OF THE INVENTION

### 1. Field of the Invention

The present invention relates to a treatment instrument insertion tool which is attached to an insertion device inserted into a subject and is configured to allow insertion of a treatment instrument configured to treat the subject into the subject.

### 2. Description of the Related Art

Conventionally, insertion devices inserted into a subject, for example, endoscopes, are widely used in a medical field, an industrial field, and the like. Among these insertion devices, medical endoscopes in the medical field are configured to be able to observe an organ or the like using an elongated tubular insertion portion inserted into a subject, for example, a body cavity of a living body, or apply various types of treatment to the organ or the like using a treatment instrument inserted into a treatment instrument insertion channel provided in the endoscope as necessary. On the other hand, industrial endoscopes used in the industrial field are configured to be able to observe or inspect a state inside an object, for example, a state of a flaw or corrosion by inserting an insertion portion into the object, for example, an apparatus such as a jet engine or pipe in a factory or machine equipment.

Thus, when observing a body cavity of a living body or the interior of machine equipment using an endoscope as an insertion device, it is a general practice that a user performs an insertion operation or the like by grasping the insertion portion of the endoscope by one hand (e.g., right hand) and grasping an operation portion of the endoscope by the other hand (e.g., left hand) to perform various operations while visually checking an endoscope image displayed on a display apparatus.

Furthermore, for example, when applying treatment or the like to a desired region in a body cavity of a living body, an operator (user) operates a treatment instrument by grasping the insertion portion and performs a desired treatment operation. In this case, the operator (user) takes the one hand (e.g., right hand) off the insertion portion, grasps the treatment instrument by the free hand (e.g., right hand) and inserts the treatment instrument into the insertion channel via an insertion port of the treatment instrument insertion channel provided at the operation portion. Then, the treatment instrument protrudes from an opening at the distal end of the insertion portion formed at a distal end of the insertion portion and communicating with the treatment instrument insertion channel. This makes it possible to perform various types of treatment using the treatment instrument.

In this case, the operator (user) makes a fine adjustment of the distal end position of the endoscope insertion portion and the distal end position of the treatment instrument with respect to a desired target region to be treated (hereinafter referred to as a “treatment target region”) while visually checking an endoscope image, and can thereby be ready to perform appropriate treatment.

Japanese Patent No. 5487343, Japanese Patent No. 5543733, International Publication No. 2013/065509 and the like propose a treatment instrument insertion tool configured to make it possible to perform a forward/backward operation of a treatment instrument while holding an insertion portion of an endoscope which is an insertion device by one hand.

The treatment instrument insertion tool disclosed in above-described Japanese Patent No. 5487343, Japanese Patent No. 5543733 and the like is configured such that a forward/backward operation auxiliary tool is attached to a treatment instrument in a flexible tube via a groove formed in the flexible tube, and the operator (user) can perform a forward/backward operation of the treatment instrument by moving the forward/backward operation auxiliary tool along the groove by the hand grasping the insertion portion of the endoscope.

The treatment instrument insertion tool disclosed in above-described International Publication No. 2013/065509 is constructed of a guide portion body, a tubular treatment instrument guide portion, connected to the guide portion body, into which a treatment instrument is inserted, and a slider portion which is an operation portion attached to the treatment instrument and configured to be attachable/detachable to/from the guide portion body, or the like. With the guide portion body attached to a region of the insertion portion located outside the object and one end of the treatment instrument guide portion attached to the insertion port of the treatment instrument insertion channel, the treatment instrument is inserted into the treatment instrument insertion channel via the treatment instrument guide portion from the guide portion body, the distal end is caused to protrude from the distal end opening of the insertion portion distal end toward the inside of the object. This configuration allows the operator (user) to grasp the insertion portion by one hand (e.g., right hand) and at the same time use the treatment instrument operation portion such as a slider fixed to the guide portion body by the same one hand (e.g., right hand) to make a positional adjustment of the treatment instrument. Thus, the operator (user) can make a fine adjustment of the respective distal end positions of the insertion portion and the treatment instrument with respect to the treatment target region using only one hand (e.g., right hand) at the same time.

### SUMMARY OF THE INVENTION

A treatment instrument insertion tool according to one aspect of the present invention is a treatment instrument insertion tool attached to an insertion portion of an insertion device inserted into a subject, configured to allow insertion of a treatment instrument into the insertion portion, including a tubular treatment instrument guide portion, an end of which is connectable to a treatment instrument insertion port of the insertion device, configured to guide the treatment instrument into the insertion portion via the treatment instrument insertion port, a guide portion body freely attachably/detachably provided in a region of the insertion portion located outside the subject when the insertion portion of the insertion device is inserted into the subject, one end of the treatment instrument guide portion being connected to the guide portion body, and the guide portion body including an insertion port of the treatment instrument with respect to the treatment instrument guide portion, and a film member fixed to one end of the guide portion body in a connection region between the one end of the treatment instrument guide portion and the guide portion body, the film member including a hole that allows insertion of the treatment instrument, water-tightly engaging with the treatment instrument when the treatment instrument is inserted into the hole, and being formed of a material freely expandable/contractible and restricting permeation of a liquid.

### BRIEF DESCRIPTION OF THE DRAWINGS

FIG. 1 is a schematic configuration diagram illustrating a state in which a treatment instrument insertion tool according to a first embodiment of the present invention is used together with an insertion device (endoscope);

FIG. 2 is a cross-sectional view of main parts illustrating a state in which a treatment instrument is inserted into the treatment instrument insertion tool in FIG. 1 and a treatment instrument operation portion is pulled toward a hand side;

FIG. 3 is a cross-sectional view of main parts illustrating a state in which a treatment instrument is inserted into the treatment instrument insertion tool in FIG. 1 and the treatment instrument operation portion is pushed toward an insertion side;

FIG. 4 is a cross-sectional view of main parts illustrating a treatment instrument insertion tool according to a second embodiment of the present invention, in a state in which a treatment instrument is inserted and a treatment instrument operation portion is pushed toward an insertion side;

FIG. 5 is a cross-sectional view of main parts illustrating the treatment instrument insertion tool according to the second embodiment of the present invention in a state in which the treatment instrument is inserted and the treatment instrument operation portion is pulled toward a hand side;

FIG. 6 is a cross-sectional view of main parts of a treatment instrument insertion tool according to a third embodiment of the present invention in a state in which a treatment instrument is inserted;

FIG. 7 is a cross-sectional view of main parts of a treatment instrument insertion tool according to a fourth embodiment of the present invention in a state in which a treatment instrument is inserted, showing a process when the treatment instrument is incorporated into the treatment instrument insertion tool;

FIG. 8 is a cross-sectional view of main parts in a state after the state in FIG. 7 in which the treatment instrument and a film member are incorporated into a predetermined region of the treatment instrument insertion tool;

FIG. 9 is a conceptual diagram illustrating a configuration example when a film member is formed into a bellows-shape;

FIG. 10 illustrates a configuration example when a freely expandable/contractible film member is provided with a expansion/contraction restricting section and is a conceptual diagram illustrating a normal state of the film member; and

FIG. 11 is a conceptual diagram illustrating a state in which an expansion/contraction force quantity is added to the film member in the state in FIG. 10, causing the film member to expand.

### DETAILED DESCRIPTION OF THE PREFERRED EMBODIMENTS

The present invention will be described according to embodiments illustrated hereinafter. Each drawing used for the following description is intended for schematic illustration, and a dimensional relationship and a scale or the like among different members may vary from one component to another in order to illustrate the components in sizes recognizable on the drawings. Therefore, the present invention is not limited only to illustrated forms with respect to quantities of components, shapes of the components, size ratios among the components, relative positional relationships among the components described in the respective drawings.

### First Embodiment

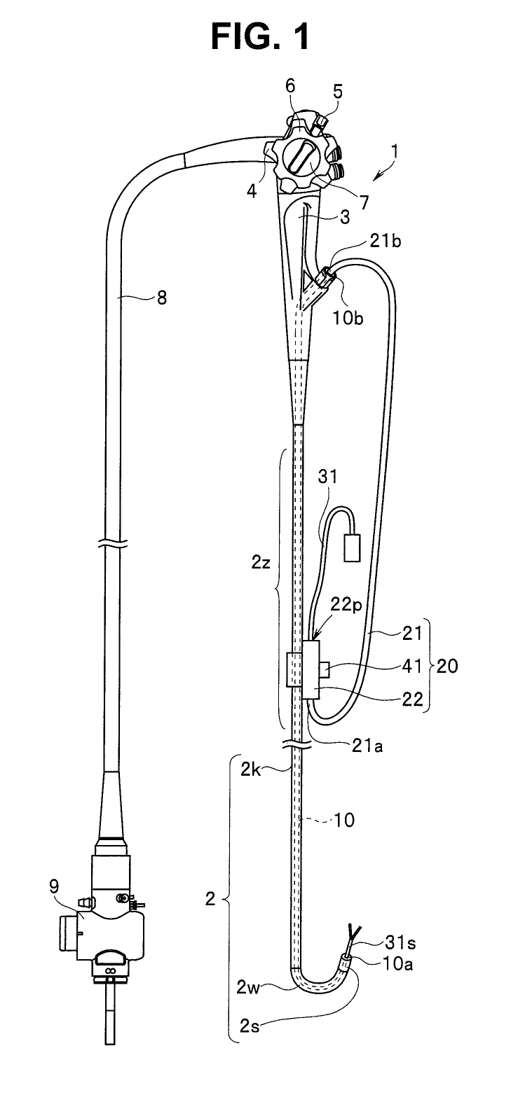

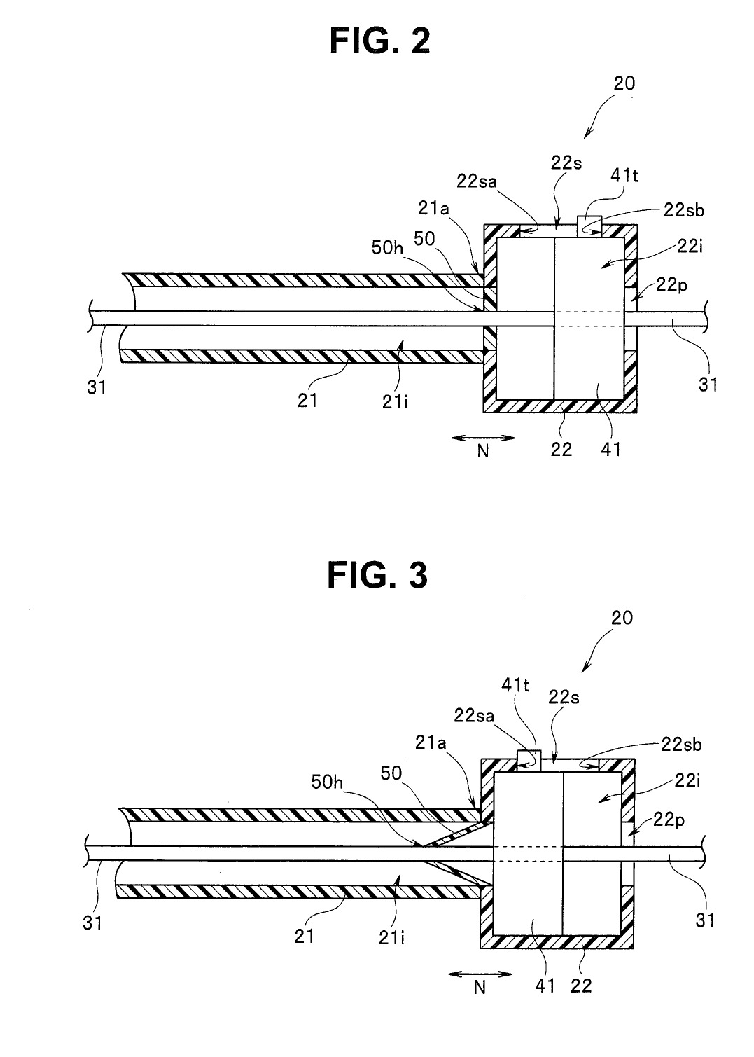

FIG. 1 is a schematic configuration diagram illustrating a state in which a treatment instrument insertion tool according to a first embodiment of the present invention is used together with an insertion device (endoscope). FIG. 2 and FIG. 3 are cross-sectional views of main parts illustrating a state in which a treatment instrument is inserted into the treatment instrument insertion tool of the present embodiment. FIG. 2 illustrates a state in which a treatment instrument operation portion is pulled toward a hand side. FIG. 3 illustrates a state in which the treatment instrument operation portion is pushed toward an insertion side.

The treatment instrument insertion tool of the present embodiment is a device attached to an insertion portion of an insertion device (e.g., endoscope) which is inserted into (e.g., body cavity) a subject (e.g., living body), configured to allow insertion of a treatment instrument into the subject via the insertion portion of the insertion device.

First, a schematic configuration of an endoscope, an insertion device, will be described, which is used together with the treatment instrument insertion tool of the present embodiment when this treatment instrument insertion tool is used.

As shown in FIG. 1, an endoscope 1, an insertion device, is constructed of an insertion portion 2 inserted into (e.g., body cavity) a subject (e.g., living body), an operation portion 3 connected on a proximal end side of the insertion portion 2, a universal cord 8 extending from the operation portion 3 and a connector 9 provided at an extended end of the universal cord 8 as main parts.

Though not shown in the drawing, the endoscope 1 is electrically connected to a control apparatus, which is not shown, or an external apparatus such as an illumination apparatus via the connector 9.

The operation portion 3 is provided with a vertical bending operation knob 4 configured to bend a bending portion 2w of the insertion portion 2 in a vertical direction and a horizontal bending operation knob 6 configured to bend the bending portion 2w in a horizontal direction.

The operation portion 3 is further provided with a fixing lever 5 configured to fix a turning position of the vertical bending operation knob 4 and a fixing knob 7 configured to fix a turning position of the horizontal bending operation knob 6. The operation portion 3 is further provided with various known operation switches.

The various knobs 4, 6 and 7, the lever 5 and the various switches provided in the operation portion 3 are operated by, for example, the operator's left hand.

The insertion portion 2 is constructed of a distal end portion 2s, the bending portion 2w and a flexible tube portion 2k in that order from the distal end side. The insertion portion 2 is formed into an elongated tube shape. The insertion portion 2 is operated, for example, by the operator's right hand.

Note that in contrast to the aforementioned case, various operations of the insertion portion 2 may be performed by the left hand and various operations of the operation portion 3 may be performed by the right hand.

The bending portion 2w is bent, for example, in four directions of upward, downward, rightward and leftward directions through turning operations of the vertical bending operation knob 4 and the horizontal bending operation knob 6. This allows the bending portion 2w to change an observation direction of an image pickup unit, which is not shown, provided in the distal end portion 2s and improve insertability of the distal end portion 2s when the insertion portion 2 is inserted into the subject. Furthermore, the flexible tube portion 2k is connected on a proximal end side of the bending portion 2w.

Furthermore, a treatment instrument insertion channel 10 is provided in the insertion portion 2 and the operation portion 3. One end of the treatment instrument insertion channel 10 is opened on a distal end face of the distal end portion 2s as a distal end opening 10a and the other end is opened at the operation portion 3 as an insertion port 10b.

The rest of the configuration of the endoscope 1 is similar to a configuration of a common endoscope, and so detailed description will be omitted.

Next, a configuration of the treatment instrument insertion tool of the present embodiment will be described below. As shown in FIG. 1, a treatment instrument insertion tool 20 is configured to allow insertion of a treatment instrument 31 attached to the endoscope 1 and configured to treat the inside of the subject into the subject (e.g., into a body cavity of a living body). The treatment instrument 31 here has a conventional form which is commonly used.

The present embodiment shows a case where the number of treatment instruments is one as an example, but the treatment instrument insertion tool 20 can be used by selectively inserting a plurality of treatment instruments into the subject.

The treatment instrument insertion tool 20 is constructed of a tube 21 which is a tubular treatment instrument guide portion, a guide portion body 22 and a slider 41 which is a treatment instrument operation portion as main parts.

The tube 21 is a tubular member configured to guide the treatment instrument 31 to the inside of the subject via the treatment instrument insertion channel 10 (inside of the insertion portion) of the endoscope 1 (insertion device). The tube 21 is formed of, for example, polytetrafluoroethylene (PTFE).

One end 21a of the tube 21 is connected to the guide portion body 22 and the other end 21b is connected to the insertion port 10b of the treatment instrument insertion channel 10 of the endoscope 1. In this condition, the treatment instrument 31 can be inserted through the guide portion body 22 and the tube 21, passed from the insertion port 10b to the treatment instrument insertion channel 10 and further caused to protrude outward from the distal end opening 10a of the distal end portion 2s of the insertion portion 2 of the endoscope 1 via the treatment instrument insertion channel 10. Thus, the treatment instrument insertion tool 20 is configured to guide the distal end portion of the treatment instrument 31 to a desired treatment target region inside the subject.

When the insertion portion 2 of the endoscope 1 (insertion device) is inserted into the subject, the guide portion body 22 is freely attachably/detachably provided in a region of the insertion portion 2 located outside the subject. In addition, one end of the tube 21 (treatment instrument guide portion) is connected to the guide portion body 22. The guide portion body 22 further includes an insertion port 22p configured to insert the treatment instrument 31 to the tube 21 (treatment instrument guide portion).

That is, one end 21a of the tube 21 is connected to the guide portion body 22 as illustrated and described above. In this way, the guide portion body 22 internally communicates with a space 21i inside the tube 21.

The guide portion body 22 also includes a space 22i where the treatment instrument 31 is inserted and the slider 41 is attached so as to move freely in a predetermined direction.

The insertion port 22p is formed in a surface on a proximal end side of the space 22i and facing a connection region of the tube 21 when the treatment instrument 31 is inserted toward the tube 21.

The guide portion body 22 is configured to be attachable/detachable to/from the insertion portion 2 of the endoscope 1. In that case, the guide portion body 22 is configured, when used together with the endoscope 1, to be attached to a region of the flexible tube portion 2k of the insertion portion 2 located outside the subject, for example, a region within a range shown by reference character 2z in FIG. 1 when the insertion portion 2 of the endoscope 1 is inserted into the subject.

In a condition in which the guide portion body 22 is attached to the predetermined region 2z located outside the subject, the guide portion body 22 is fixed with respect to the insertion portion 2 using predetermined means so that the condition is always maintained, that is, the attachment position of the guide portion body 22 with respect to the insertion portion 2 is not shifted. In this case, the guide portion body 22 may also be configured to be attachable/detachable to/from the insertion portion 2.

Furthermore, as shown in FIG. 2 and FIG. 3, a slit 22s configured to communicate with the space 22i is formed in the guide portion body 22. The slit 22s is formed in the guide portion body 22 along a longitudinal direction N in which the tube 21 guides the treatment instrument 31. Though details will be described later, the slit 22s is a region that functions as a movement restricting section that restricts the moving range of the slider 41 which is a treatment instrument operation portion.

The slider 41 is a treatment instrument operation member which is formed into a cylindrical shape and attached to the treatment instrument 31. The slider 41 is formed to be freely attachable/detachable to/from the guide portion body 22.

That is, the slider 41 is a component attached to the treatment instrument 31 in advance. The slider 41 is provided to be attachable/detachable to/from the space 22i of the guide portion body 22. After being attached to the space 22i of the guide portion body 22, the slider 41 moves forward/backward in a longitudinal direction relative to the tube 21 and integrally with the treatment instrument 31. The slider 41 may also be attachable/detachable to/from the treatment instrument 31.

Note that the slider 41 is attached to a predetermined region on the proximal end side of the treatment instrument 31, that is, a predetermined region closer to an end on a side opposite to the distal end 31s of the treatment instrument 31 (see FIG. 1). More specifically, the position at which the slider 41 is attached to the treatment instrument 31 is a predetermined position at which it is possible to insert the treatment instrument 31 through the treatment instrument insertion tool 20 and the insertion portion 2 of the endoscope 1 and cause the distal end 31s of the treatment instrument 31 to protrude outward from the distal end opening 10a of the distal end portion 2s of the insertion portion 2.

As shown in FIG. 2 and FIG. 3, the slider 41 includes a protrusion 41t on an outer surface. While the slider 41 is housed in the space 22i, the protrusion 41t engages with the slit 22s and is slidably disposed in a direction along the slit 22s. Thus, the slider 41 is configured to be able to move in a direction along the longitudinal direction N in the space 22i of the guide portion body 22 while it is attached to the treatment instrument 31. Note that the protrusion 41t may be formed integrally with the slider 41. Furthermore, a protrusion member formed separately from the slider 41 may be configured to be attachable/detachable to/from the outer surface of the slider 41. In this case, means like a screwing mechanism or an engagement mechanism may be used, in which a female screw hole or an engagement hole is formed on the outer surface of the slider 41 and a male screw portion or an engagement bar-shaped portion is formed on a side of the separate member corresponding to the protrusion 41t.

That is, the protrusion 41t moves forward/backward in the longitudinal direction N between a distal end 22sa and a proximal end 22sb of the slit 22s. Along with this, the slider 41 moves in the same direction in the space 22i of the guide portion body 22. Therefore, the slit 22s and the protrusion 41t restrict the moving range of the slider 41 and thereby function as a movement restricting section configured to restrict a range in which the treatment instrument 31 moves together with the slider 41.

Note that in a state in which the slider 41 is attached to the guide portion body 22, the treatment instrument 31 is inserted into the space 21i and the space 22i, and the slider 41 is disposed in the space 22i and the protrusion 41t engages with the slit 22s. In this state, as shown in FIG. 1, the distal end 31s of the treatment instrument 31 protrudes from the distal end opening 10a at the distal end portion 2s of the insertion portion 2 of the endoscope 1.

Furthermore, as shown in FIG. 2, a hole 50h is formed at substantially a central part in the vicinity of a connection region which becomes a boundary between the space 21i of the tube 21 and the space 22i of the guide portion body 22 to allow the treatment instrument 31 to pass and a substantially circular thin planar film member 50 is provided so as to cover the portion communicating between both spaces 21i and 22i.

The film member 50 is fixed to one end of the guide portion body 22 in the connection region between the tube 21 (treatment instrument guide portion) and the guide portion body 22, includes the hole 50h that allows insertion of the treatment instrument 31, and is formed of a material that water-tightly engages with the treatment instrument 31 inserted through the hole 50h, is freely expandable/contractible and prevents permeation of a liquid. The hole 50h of the film member 50 constitutes an engaging portion that allows the treatment instrument 31 to be inserted and allows the treatment instrument 31 and the film member 50 to engage with each other water-tightly and liquid-tightly.

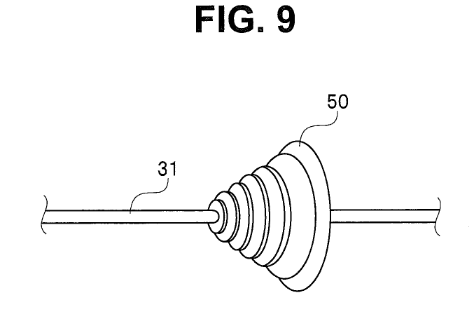

That is, this film member 50 is formed using a material which is a flexible thin film to allow free expansion/contraction, and has water-tightness or liquid-tightness. Note that the film member 50 may be formed into, for example, a bellows shape as shown in FIG. 9 to acquire a freely expandable/contractible form instead of using a thin planar expandable/contractible material. In this way, when the film member 50 is formed into a bellows shape, the material itself that forms the film member 50 need not be made of an expandable/contractible material.

An outer peripheral edge of the film member 50 is bonded/fixed to a fixing portion of the guide portion body 22, for example. When the slider 41 to which the treatment instrument 31 is attached is attached to a predetermined region in the guide portion body 22, the treatment instrument 31 is disposed by passing through the hole 50h of the film member 50. At this time, the hole 50h of the film member 50 and the outer circumferential surface of the treatment instrument 31 are brought into close contact with each other and thereby configured to secure water tightness or liquid tightness.

For this reason, the hole 50h of the film member 50 is formed to have a size which is sufficiently smaller than a maximum size of the cross section of the treatment instrument 31. That is, more specifically, when, for example, the hole 50h is formed into a circular shape and the treatment instrument 31 is formed to have a substantially circular cross-sectional shape, the hole 50h has a diameter sufficiently smaller than the diameter of the circular cross section of the treatment instrument 31. As another form of the hole 50h, for example, a cross-like notch may be provided in a substantially central part of the film member 50. In this case, the size of the cross-like notch is made sufficiently smaller than a maximum size of the cross section of the treatment instrument 31.

In the above-described condition, that is, when the slider 41 to which the treatment instrument 31 is attached is attached to a predetermined region in the guide portion body 22 and the treatment instrument 31 is inserted through the hole 50h of the film member 50, the film member 50 and the treatment instrument 31 are bonded and fixed to each other at a closely contacted region between the hole 50h and the outer circumferential surface of the treatment instrument 31.

As described above, the treatment instrument 31 is integrally attached to the slider 41. Thus, when the slider 41 moves forward/backward in the longitudinal direction N along the slit 22s, the treatment instrument 31 also moves forward/backward in the same direction. In such a configuration, when the treatment instrument 31 moves forward/backward along the longitudinal direction N, which is an axial direction of the treatment instrument 31, the film member 50 flexibly expands/contracts following the forward/backward movement of the treatment instrument 31. In this case, if the amount of forward/backward movement along the longitudinal direction N of the slider 41 and the treatment instrument 31 is large, the film member 50 that expands/contracts following this may be damaged when an allowable expansion/contraction limit is surpassed. However, as described above, the forward/backward movement range in the same direction of the slider 41 is restricted by the slit 22s. This prevents the film member 50 from going beyond the allowable expansion/contraction limit and being damaged. That is, the above-described slit 22s and protrusion 41t also play a role as a deformation amount restricting section configured to restrict the amount of deformation when the film member 50 expands/contracts in the longitudinal direction of the treatment instrument 31 and is deformed. Note that the amount of forward/backward movement of the treatment instrument 31 performed to make a fine adjustment of the position of the distal end of the treatment instrument 31 in the body cavity is not so large, which falls well within the allowable expansion/contraction range of the film member 50, and so it is possible to fully make a desired fine adjustment, without posing any problem.

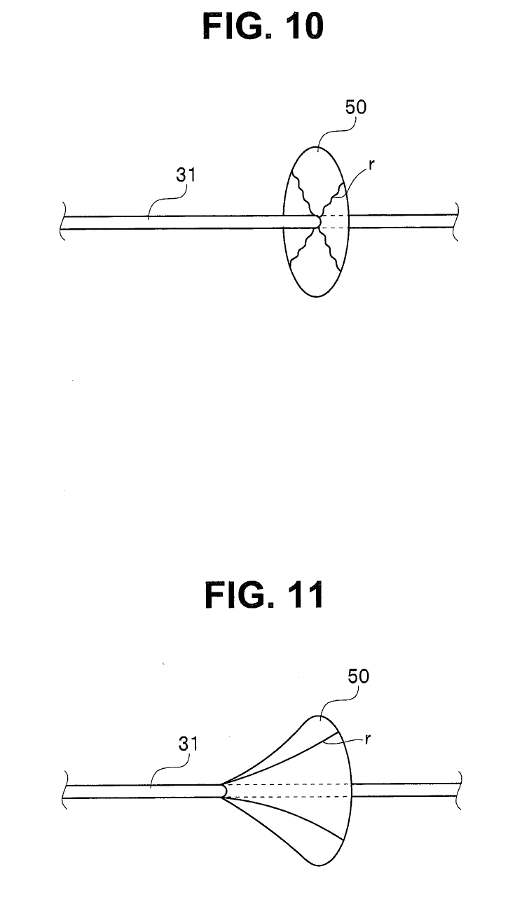

The film member 50 plays a role to prevent a liquid such as a body fluid in the body cavity which flows reversely to the space 22i of the guide portion body 22 via the treatment instrument insertion channel 10 of the endoscope 1 and the space 21i of the tube 21 from leaking outside. Note that when the film member 50 is formed of a thin planar freely expandable/contractible material, an expansion/contraction restricting section r for restricting expansion/contraction may be provided in some region of the film member 50 as shown in FIG. 10. FIG. 10 and FIG. 11 are conceptual diagrams illustrating a configuration example when the expansion/contraction restricting section is provided in the freely expandable/contractible film member. Note that FIG. 10 illustrates a normal state in which no expansion/contraction force is applied to the film member. FIG. 11 illustrates a state in which the film member is expanded. The film member 50 in such a form, that is, the film member 50 formed using a freely expandable/contractible material is provided with the expansion/contraction restricting section r formed in a part of its region using, for example, a non-expandable fibrous material. When such a configuration is adopted, it is possible to easily restrict the expansion/contraction range of the film member 50 as shown in FIG. 11, and so it is possible to prevent the water-tightness or liquid-tightness of the film member 50 from being unintentionally broken when the film member 50 is stretched excessively or prevent the film member 50 from being damaged.

An overview of operation when using the treatment instrument insertion tool 20 of the present embodiment configured in this way together with the endoscope 1, an insertion device, will be described below. Note that although terms such as “first” and “next” are used for convenience in the following description, the described order need not always be followed, and the order may be reversed.

First, when a predetermined region of the insertion portion 2 of the endoscope 1, that is, the insertion portion 2 is inserted for use into the body cavity of the subject, the guide portion body 22 of the treatment instrument insertion tool 20 is attached to the region 2z located outside the subject.

Next, the tube 21 which is the treatment instrument guide portion of the treatment instrument insertion tool 20 is connected to the insertion port 10b of the treatment instrument insertion channel 10 of the endoscope 1.

Furthermore, the slider 41, which is a treatment instrument operation portion, is attached to a predetermined region of the treatment instrument 31. The position at which the treatment instrument 31 is attached to the slider 41 is a predetermined position at which when, for example, the treatment instrument 31 is inserted through the treatment instrument insertion tool 20 and the insertion portion 2 of the endoscope 1, the distal end 31s of the treatment instrument 31 protrudes outward from the distal end opening 10a of the distal end portion 2s of the insertion portion 2 of the endoscope 1. The amount of protrusion of the distal end 31s of the treatment instrument 31 from the distal end opening 10a in this case is defined as appropriate depending on the region to be the target of treatment and manipulation employed or the like.

Here, the treatment instrument 31 to which the slider 41 is attached in advance is inserted into the treatment instrument insertion channel 10 of the endoscope 1 via the treatment instrument insertion tool 20. That is, the tubular portion (insertion portion) of the treatment instrument 31 is inserted from the insertion port 22p of the guide portion body 22 into the space 22i inside the guide portion body 22. The tubular portion (insertion portion) of the treatment instrument 31 is pushed as it is so as to pass through the hole 50h. When the tubular portion (insertion portion) of the treatment instrument 31 is further pushed in, the treatment instrument 31 is inserted into the space 21i of the tube 21. Furthermore, the treatment instrument 31 is inserted from the insertion port 10b into the treatment instrument insertion channel 10 and protrudes outward from the distal end opening 10a after passing through the treatment instrument insertion channel 10. When the distal end 31s of the treatment instrument 31 protrudes by a predetermined amount, the slider 41 is fixed to the treatment instrument 31. At the same time, the closely contacted region of the treatment instrument 31 and the hole 50h is bonded and fixed.

When treatment is conducted using the treatment instrument insertion tool 20, the endoscope 1 and the treatment instrument 31 configured in this way, the following operation is performed.

The operator (user) grasps the vicinity of the region to which the treatment instrument insertion tool 20 of the insertion portion 2 is attached using one hand (e.g., right hand) while observing an endoscope image displayed on a display apparatus (not shown), places a finger of the same right hand (e.g., thumb) at the protrusion 41t of the slider 41 of the treatment instrument insertion tool 20 and pushes or pulls the protrusion 41t. Note that the other hand (e.g., left hand) is grasping the operation portion 3 of the endoscope 1 at this time, ready to operate the operation member of the operation portion 3.

Such an operation causes the protrusion 41t to move forward/backward between the distal end 22sa and the proximal end 22sb along the longitudinal direction N in the slit 22s. Since the slider 41 in which the protrusion 41t is provided is integrated with the treatment instrument 31, the treatment instrument 31 also moves forward/backward by the same amount in the same direction. In this way, a fine adjustment is made to the position of the distal end 31s of the treatment instrument 31 protruding from the distal end opening 10a at the distal end portion 2s of the insertion portion 2 in the subject.

While performing a forward/backward operation of the treatment instrument 31 using one hand (right hand in this case), the operator (user) grasps the insertion portion 2 using the same one hand (right hand). Therefore, simultaneously with the operation of the treatment instrument 31, the operator (user) pushes or pulls the insertion portion 2 itself grasped using the same hand (right hand) while observing an endoscope image, and can thereby make a fine adjustment of the position of the distal end portion 2s of the insertion portion 2 in the body cavity.

Furthermore, since the operator (user) always keeps grasping the insertion portion 2 without leaving the hand from it, and so it is possible to display always stable endoscope images without worries about the distal end portion 2s of the insertion portion 2 being shifted in the body cavity.

While such a treatment operation is being performed, not only the pressure in the body cavity is higher than an outside air pressure, but also the treatment instrument 31 which passes through the treatment instrument insertion channel 10 and the tube 21 moves forward/backward. Therefore, a liquid such as a body fluid in the body cavity flowing reversely from the distal end opening 10a at the distal end portion 2s of the insertion portion 2 of the endoscope 1 indwelling in the body cavity via the treatment instrument insertion channel 10 of the endoscope 1 and the space 21i of the tube 21 of the treatment instrument insertion tool 20 caused by such an operating environment is blocked by the film member 50 provided in the treatment instrument insertion tool 20. Therefore, such a liquid will never leak from the proximal end side of the treatment instrument insertion tool 20, that is, the insertion port 22p on the hand side.

As described above, according to the first embodiment, the film member 50 made of a material flexible so as to be freely expandable/contractible and having water tightness or liquid tightness is provided on a boundary between the one end 21a of the treatment instrument guide portion (tube 21) and the guide portion body 22 in the treatment instrument insertion tool 20, and with the treatment instrument 31 being inserted through the hole 50h provided in the film member 50, the outer circumferential surface of the treatment instrument 31 and the film member 50 are fixed to each other in close contact with each other. The range of forward/backward movement in the axial direction (longitudinal direction N) of the treatment instrument 31 is restricted by restricting the movement of the treatment instrument operation portion (slider 41) in the same direction.

The film member 50 in such a configuration expands/contracts following the forward/backward movement of the treatment instrument 31, and can thereby prevent a backflow of a body fluid or the like in the body cavity while always securing water tightness or liquid tightness in the treatment instrument insertion tool 20. Simultaneously with this, it is also possible to acquire always good slidability without detriment to a forward/backward operation of the treatment instrument 31 inside the treatment instrument insertion tool 20.

Note that although the present embodiment shows an example where the film member 50 is disposed in the vicinity of the connection region which constitutes a boundary between the space 21i of the tube 21 and the space 22i of the guide portion body 22, the region in which the film member 50 is disposed is not limited to this. For example, the film member 50 may also be disposed in a region covering the insertion port 22p on the hand side of the guide portion body 22. In this case, however, a liquid such as a body fluid in the body cavity may flow reversely up to the inside of the space 22i of the guide portion body 22. Therefore, the guide portion body 22 itself is preferably provided with water tightness or liquid tightness.

### Second Embodiment

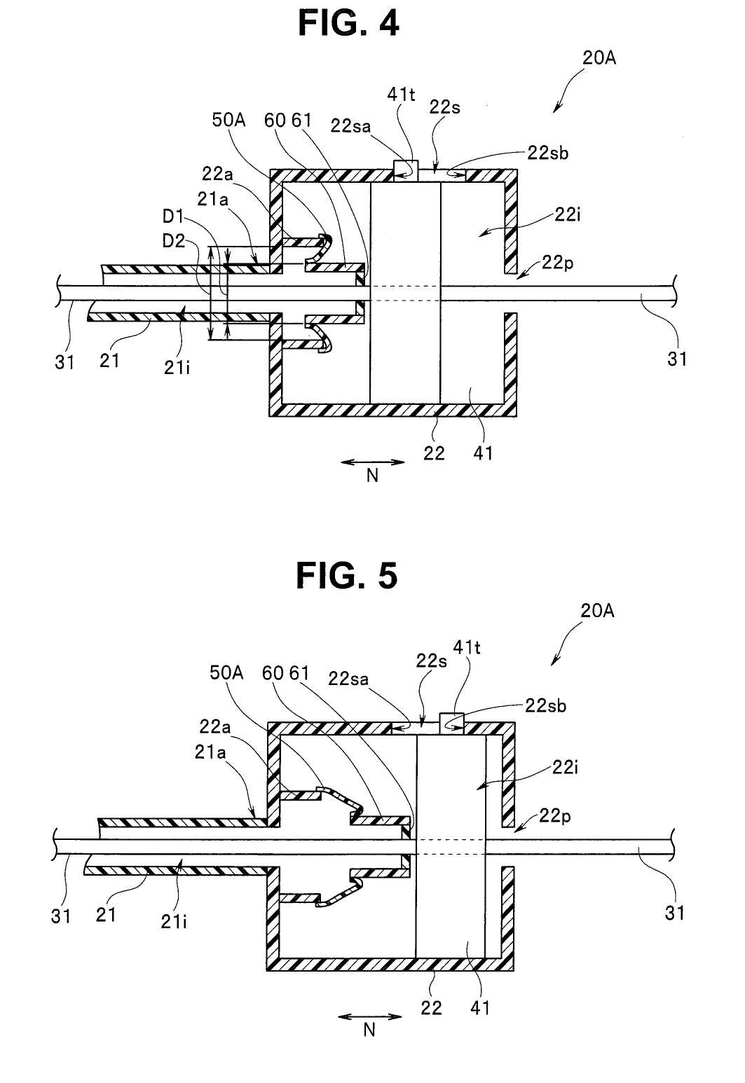

Next, a treatment instrument insertion tool according to a second embodiment of the present invention will be described using FIG. 4 and FIG. 5. FIG. 4 and FIG. 5 are cross-sectional views of main parts of the treatment instrument insertion tool according to the second embodiment of the present invention in a state in which the treatment instrument is inserted. FIG. 4 illustrates a state in which the treatment instrument operation portion is pushed toward the insertion side. FIG. 5 illustrates a state in which the treatment instrument operation portion is pulled toward the hand side.

The basic configuration of the treatment instrument insertion tool 20A of the present embodiment is substantially the same as the configuration of the aforementioned treatment instrument insertion tool 20 of the first embodiment. In the present embodiment, a film member 50A has a slightly different form in which the film member 50A is provided in the vicinity of a connection region which constitutes a boundary between the space 21i of the tube 21 and the space 22i of the guide portion body 22 and is provided so as to cover a part communicating between both the spaces 21i and 22i. The rest of the configuration is substantially the same.

That is, as shown in FIG. 4 and FIG. 5, the film member 50A is formed into a substantially cylindrical shape and is formed of a material which is flexible to be freely expandable/contractible and having water tightness or liquid tightness as in the case of the above-described first embodiment. Note that it is better that a highly self-lubricating material be applied to the above-described film member 50A in addition to the above-described condition.

One end of the film member 50A is formed, for example, inside the guide portion body 22 and bonded and fixed to a substantially cylindrical fixing portion 22a. The other end of the film member 50A is bonded and fixed to an outer peripheral edge on a distal end side of a cylindrical member 60. In this way, the film member 50A secures water tightness or liquid tightness between the space 22i of the guide portion body 22 and the space 21i of the tube 21, and water-tightly engages with the treatment instrument 31 via the cylindrical member 60 as will be described later.

Note that an outer diameter (reference character D1 in FIG. 4) of the cylindrical member 60 is smaller than an inner diameter (reference character D2 in FIG. 4) of the fixing portion of the guide portion body 22, and a difference between the outer diameter D1 of the cylindrical member 60 and the inner diameter D2 of the fixing portion of the guide portion body 22 is configured to be greater than at least double the film thickness of the film member 50.

The above-described cylindrical member 60 is disposed so as to extend parallel to the longitudinal direction of the slider 41 (treatment instrument operation portion) inside the guide portion body 22. The cylindrical member 60 is water-tightly fixed to the treatment instrument 31 and moves integrally with the treatment instrument 31.

That is, the cylindrical member 60 is a substantially cylindrical component having an opening at one end and a fixing member 61 including a hole that allows insertion of the treatment instrument 31 at the other end.

The above-described fixing member 61 is formed so as to cover the other end of the above-described cylindrical member 60 and is provided so as to fix the cylindrical member 60 to a predetermined region of the treatment instrument 31. Therefore, the above-described fixing member 61 is bonded and fixed to a predetermined region on the outer circumferential surface of the treatment instrument 31, that is, a region in which the slider 41 is housed in the guide portion body 22 in the vicinity of the region of the treatment instrument 31 to which the above-described slider 41 is fixed.

That is, in the present embodiment, the film member 50A is disposed between the treatment instrument 31 and the fixing portion 22a of the guide portion body 22 via the cylindrical member 60 fixed to the outer circumferential surface of the treatment instrument 31. In this case, the fixing member 61 and the cylindrical member 60 is fixed so as to have water tightness or liquid tightness. Furthermore, the cylindrical member 60 and the other end of the film member 50A is also bonded and fixed so as to have water tightness or liquid tightness. Furthermore, the one end of the film member 50A and the fixing portion 22a is also bonded and fixed so as to have water tightness or liquid tightness.

With the treatment instrument insertion tool 20A of the present embodiment in such a configuration, when the protrusion 41t of the slider 41 is moved forward/backward by the operator (user) in a predetermined direction, that is, in a direction along the slit 22s (along the longitudinal direction N), the treatment instrument 31 also moves in the same direction along with movement of the slider 41. A transition takes place from, for example, the state shown in FIG. 4 to a state shown in FIG. 5. That is, when the slider 41 and the treatment instrument 31 make a transition from the state in FIG. 4 to the state in FIG. 5 as described above, the cylindrical member 60 also moves in the same direction by the same amount accordingly. At this time, the film member 50A expands/contracts, and thereby always secures water tightness or liquid tightness between the space 21i of the tube 21 and the space 22i of the guide portion body 22. Note that the forward/backward movement of the slider 41 in the longitudinal direction N is restricted to within a predetermined range by the protrusion 41t coming into contact with the distal end 22sa and the proximal end 22sb of the slit 22s as in the case of the first embodiment. Note that the rest of the configuration of the treatment instrument insertion tool 20A of the present embodiment is similar to the configuration of the aforementioned embodiment, and so illustration and detailed description of those components will be omitted.

As described above, according to the above-described second embodiment, it is possible to obtain substantially the same effects as the effects of the first embodiment. Interposition of the cylindrical member 60 between the fixing portion 22a of the guide portion body 22 and the treatment instrument 31 in the treatment instrument insertion tool 20A makes it possible to secure the amount of movement to make a greater adjustment. In this case, it is possible to further improve slidability of the treatment instrument 31 by forming the above-described film member 50A using a higher self-lubricating material.

Furthermore, the cylindrical member 60 is arranged so as to move within the space 22i of the guide portion body 22, but since the cylindrical member 60 is configured to seal the inner space on the tube 21 side of the cylindrical member 60, a liquid such as a body fluid in the body cavity flowing reversely never leaks inside the space 22i of the guide portion body 22. In this case, the reversely flowing liquid such as a body fluid in the body cavity is received in the space enclosed by the cylindrical member 60, the film member 50A and the fixing portion 22a of the guide portion body 22, that is, the space connected to the space 21i of the tube 21, and so there is no worry about the liquid being leaked outside.

### Third Embodiment

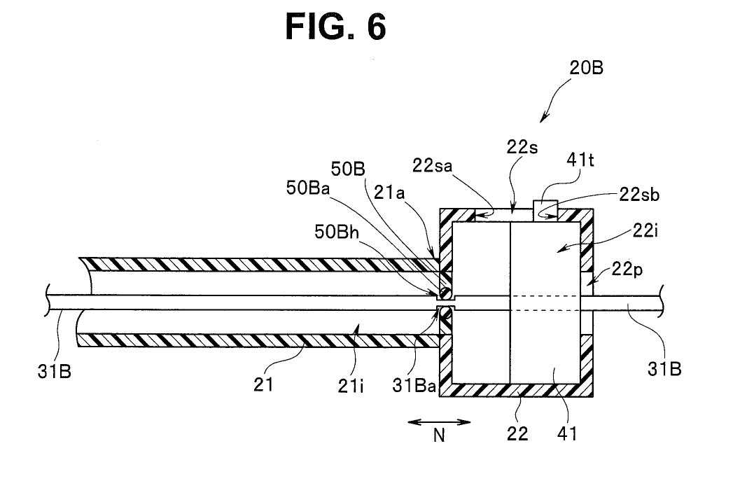

Next, a treatment instrument insertion tool according to a third embodiment of the present invention will be described below using FIG. 6. FIG. 6 is a cross-sectional view of main parts of the treatment instrument insertion tool according to the third embodiment of the present invention in a state in which a treatment instrument is inserted.

The basic configuration of the treatment instrument insertion tool 20B of the present embodiment is substantially the same as the configuration of the treatment instrument insertion tool 20 of the aforementioned first embodiment. The present embodiment is different in that an annular fixing portion 50Ba is formed like an O-shaped ring at a peripheral edge of a hole 50Bh of a film member 50B, comes into close contact with the treatment instrument 31 and engages with the treatment instrument 31.

The present embodiment is also different in that in correspondence with this, a concave circumferential groove 31Ba with which the above-described annular fixing portion 50Ba engages is formed in a predetermined region on the outer circumferential surface of the treatment instrument 31B used for the treatment instrument insertion tool 20B. The rest of the configuration is substantially the same as the configuration of the aforementioned first embodiment.

Operation when inserting the treatment instrument 31B into the body cavity of a desired subject via the endoscope 1 using the treatment instrument insertion tool 20B of the present embodiment configured in this way is as follows.

First, the tubular portion (insertion portion) of the treatment instrument 31B is inserted from the insertion port 22p of the guide portion body 22 into the space 22i inside the guide portion body 22. The tubular portion (insertion portion) of the treatment instrument 31B is pushed in as it is and caused to pass through the hole 50Bh. The tubular portion (insertion portion) of the treatment instrument 31B is further pushed in and the treatment instrument 31B is caused to be inserted into the space 21i of the tube 21. The treatment instrument 31B is then inserted from the insertion port 10b into the treatment instrument insertion channel 10 and protrudes outward from the distal end opening 10a after passing through the treatment instrument insertion channel 10. Thus, when the distal end 31s of the treatment instrument 31B protrudes by a predetermined amount, that is, when the treatment instrument 31B is arranged at a defined position, the annular fixing portion 50Ba of the film member 50B is fitted into the circumferential groove 31Ba of the treatment instrument 31B, thus engaged with the circumferential groove 31Ba, brought into close contact with and fixed to the circumferential groove 31Ba. Therefore, in this case, the above-described annular fixing portion 50Ba constitutes an engaging part that engages with the circumferential groove 31Ba on the outer circumferential surface of the treatment instrument 31B.

Therefore, according to the present embodiment, when the annular fixing portion 50Ba of the film member 50B engages with the circumferential groove 31Ba of the treatment instrument 31B, the treatment instrument 31B is automatically disposed at a predetermined position.

As described above, the above-described third embodiment can obtain effects similar to the effects of the aforementioned first embodiment. In addition, the present embodiment further uses the treatment instrument 31B including the corresponding circumferential groove 31Ba, and can thereby easily keep a closely contacted state without bonding and fixing the engagement region between the treatment instrument 31B and the hole 50Bh of the film member 50B and reliably fix the film member 50B. At the time point at which the circumferential groove 30Ba engages with the hole 50Bh of the film member 50B, the treatment instrument 31B can be automatically set at the position at which it should be disposed.

Note that the region in which the film member 50B is disposed is not limited to the aforementioned example in the present embodiment either, and the film member 50B may be disposed in a region covering the insertion port 22p on the hand side of the guide portion body 22 while securing water tightness or liquid tightness of the guide portion body 22 itself similarly to the above-described first embodiment.

### Fourth Embodiment

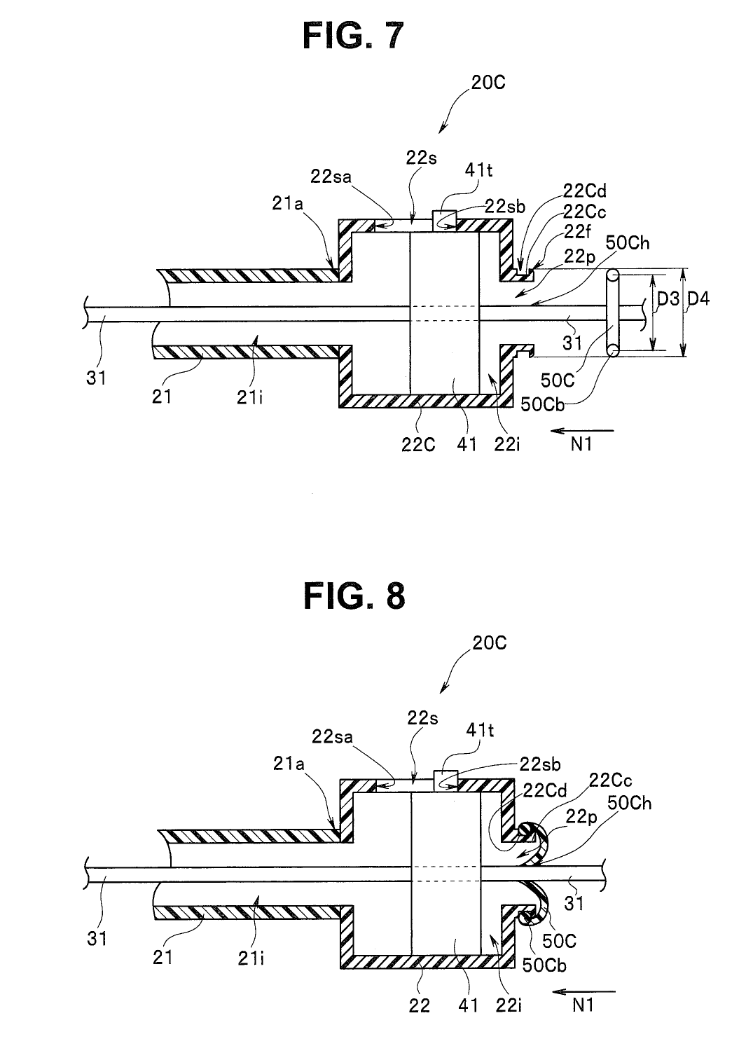

Next, a treatment instrument insertion tool according to a fourth embodiment of the present invention will be described below using FIG. 7 and FIG. 8. FIG. 7 and FIG. 8 are cross-sectional views of main parts of the treatment instrument insertion tool according to the fourth embodiment of the present invention in a state in which the treatment instrument is inserted. FIG. 7 illustrates a process in which the treatment instrument is incorporated into the treatment instrument insertion tool of the present embodiment. FIG. 8 illustrates a state in which the treatment instrument and a film member are incorporated in a predetermined region of the treatment instrument insertion tool of the present embodiment and are ready to be used.

The treatment instrument insertion tool 20C of the present embodiment basically has substantially the same configuration as the configurations of the aforementioned treatment instrument insertion tools 20 and 20B of the first and third embodiments. A difference lies in that the present embodiment is configured such that an annular fixing portion 50Cb like an O-shaped ring is formed on an outer peripheral edge of a film member 50C and a hole is provided at a substantially central part to allow insertion of the treatment instrument 31 in close contact. Note that the present embodiment is similar in that the film member 50C is formed using a material which is substantially circular, thin, planar and flexible to be freely expandable/contractible and having water tightness or liquid tightness.

The present embodiment is also different in a configuration in which when the treatment instrument insertion tool 20C is assembled, the film member 50C is disposed in a region covering the insertion port 22p on the hand side of the guide portion body 22C. In this case, according to the present embodiment, a fixing portion 22Cc provided with a circumferential groove 22Cd to engage with the annular fixing portion 50Cb of the film member 50C is formed in the vicinity of an outer peripheral edge of the insertion port 22p on the hand side of the guide portion body 22C. In this case, as shown in FIG. 7, a size D4 on the outer circumferential side of the above-described fixing portion 22Cc is set to be slightly greater than a size D3 on the outer circumferential side of the film member 50C (D3<D4).

When the annular fixing portion 50Cb of the film member 50C is caused to engage with the circumferential groove 22Cd of the fixing portion 22Cc, the annular fixing portion 50Cb is brought into engagement with the circumferential groove 22Cd while stretching the film member 50C. The fixing portion 22Cc and the film member 50C are then brought into a closely contacted state.

That is, the film member 50C is detachably attached to the fixing portion 22Cc which is one end of the guide portion body 22C. Thus, the peripheral portion of the film member 50C constitutes an elastic portion that expands/contracts so as to water-tightly engage with the outer circumference of the fixing portion 22Cc of the guide portion body 22C. When the treatment instrument 31 moves in a direction along the longitudinal direction N, the film member 50C expands/contracts in the same direction together with the treatment instrument 31.

Therefore, the present embodiment is configured to always keep water tightness or liquid tightness between the outside of the guide portion body 22 and the space 22i inside. The rest of the configuration is substantially the same as the configurations of the aforementioned first and third embodiments. Note that the treatment instrument 31 used for the treatment instrument insertion tool 20C of the present embodiment has a conventional form which is commonly used.

Operation of attaching the treatment instrument 31 to the treatment instrument insertion tool 20C of the present embodiment configured in this way is as follows.

First, the slider 41 and the film member 50C are attached at a predetermined position of the tubular portion (insertion portion) of the treatment instrument 31 in advance.

Next, a distal end of the tubular portion (insertion portion) of the treatment instrument 31 is inserted from the insertion port 22p of the guide portion body 22 into the space 22i inside the guide portion body 22. The tubular portion (insertion portion) of the treatment instrument 31 is then pushed as it is in a direction shown by an arrow N1 in FIG. 7 and FIG. 8 and the treatment instrument 31 is inserted into the space 21i of the tube 21. The treatment instrument 31 is further pushed in the same direction and disposed at a predetermined position at which the distal end of the treatment instrument 31 protrudes outward by a predetermined amount from the distal end opening 10a of the endoscope insertion portion from the insertion port 10b of the above-described endoscope insertion portion after passing through the treatment instrument insertion channel 10. The position of the treatment instrument 31 at this time becomes a defined position. That is, in this condition, the annular fixing portion 50Cb of the film member 50C is brought into engagement with the circumferential groove 22Cd provided in the fixing portion 22Cc of the guide portion body 22C. This causes the film member 50C to be fixed to the guide portion body 22 in close contact.

Note that in this case, when the film member 50C is attached so as to cover the insertion port 22p of the guide portion body 22, part of the film member 50C is disposed along the outer surface of the fixing portion 22Cc of the guide portion body 22. Since the film member 50C is formed into a thin-film shape, for example, when the film member 50C is rubbed with corners or the like of the outer surface of the guide portion body 22, it may be damaged. In consideration of this, the corners of the outer surface of the fixing portion 22Cc in the guide portion body 22 in particular (e.g., region indicated by reference character 22f in FIG. 7) are preferably subjected to chamfering or R chamfering.

As described above, the above-described fourth embodiment can obtain effects similar to the effects of the above-described first and third embodiments. In addition, according to the present embodiment, the concave groove-like circumferential groove 22Cd is provided on the side of the fixing portion 22Cc of the guide portion body 22C and the annular fixing portion 50Cb is formed on the outer circumferential side of the film member 50C in correspondence with this. After attaching this film member 50C to the treatment instrument 31 in advance, the treatment instrument 31 may be incorporated into the guide portion body 22C of the treatment instrument insertion tool 20C. Therefore, according to the present embodiment, it is possible to contribute to simplification of work of attaching the treatment instrument 31 to the treatment instrument insertion tool 20C.

Note that the present invention is not limited to the aforementioned embodiments, and it goes without saying that various modifications and applications can be made without departing from the spirit and scope of the present invention. Furthermore, the above-described embodiments include inventions at various stages, and various inventions can be extracted depending on appropriate combinations based on a plurality of disclosed configuration elements. The problems to be solved by the invention can be solved even when, for example, some of all the configuration elements shown in the above-described embodiment are deleted, and the configuration from which these configuration elements are deleted can be extracted as the invention in a case where effects of the invention can be obtained. Moreover, components over different embodiments may be combined as appropriate. This invention is not restricted by specific embodiments except in that it is restricted by the appended claims.

The present invention is applicable not only to an endoscope control apparatus in the medical field but also to an endoscope control apparatus in the industrial field.

What is claimed is:

1. A treatment instrument insertion tool attached to an insertion portion of an insertion device inserted into a subject, configured to allow insertion of a treatment instrument into the insertion portion, the treatment instrument insertion tool comprising:
a tubular treatment instrument guide portion, an end of which is connectable to a treatment instrument insertion port of the insertion device, configured to guide the treatment instrument into the insertion portion via the treatment instrument insertion port;
a guide portion body freely attachably/detachably provided in a region of the insertion portion located outside the subject when the insertion portion of the insertion device is inserted into the subject, one end of the treatment instrument guide portion being connected to the guide portion body, and the guide portion body comprising an insertion port of the treatment instrument with respect to the treatment instrument guide portion; and
a film member fixed to one end of the guide portion body in a connection region between the one end of the treatment instrument guide portion and the guide portion body, the film member comprising a hole that allows insertion of the treatment instrument, water-tightly engaging with the treatment instrument when the treatment instrument is inserted into the hole, and being formed of a material freely expandable/contractible and restricting permeation of a liquid.

2. The treatment instrument insertion tool according to claim 1, further comprising a deformation amount restricting section configured to restrict an amount of deformation when the film member is deformed in a longitudinal direction of the treatment instrument.

3. The treatment instrument insertion tool according to claim 1, wherein the hole of the film member that allows insertion of the treatment instrument is fixed to an outer circumferential surface of the treatment instrument in a closely contacted state.

4. The treatment instrument insertion tool according to claim 1, further comprising a cylindrical treatment instrument operation portion attached to the treatment instrument and formed to be freely attachable/detachable to/from the guide portion body.

5. The treatment instrument insertion tool according to claim 4, further comprising a cylindrical member extending parallel to a longitudinal direction of the treatment instrument operation portion and configured to move integrally with the treatment instrument water-tightly fixed to the treatment instrument,
wherein the film member has a cylindrical shape, one end of which is fixed to the guide portion body and another end of which is fixed to the cylindrical member, whereby the film member water-tightly engages with the treatment instrument.

6. The treatment instrument insertion tool according to claim 5,
wherein an outer diameter of the cylindrical member is smaller than an inner diameter of a fixing portion of the guide portion body, and
a difference between the outer diameter of the cylindrical member and the inner diameter of the fixing portion of the guide portion body is greater than at least double a film thickness of the film member.

7. The treatment instrument insertion tool according to claim 4,
wherein the treatment instrument operation portion further comprises a movement restricting section configured to restrict a range in which the treatment instrument moves along with the treatment instrument operation portion.

8. The treatment instrument insertion tool according to claim 1, further comprising a treatment instrument, an outer circumferential surface of which includes a circumferential groove into which an engaging part is fitted.

9. The treatment instrument insertion tool according to claim 1,
wherein the film member is detachably attached at one end of the guide portion body, and
an elastic portion configured to water-tightly engage with an outer circumference of the guide portion body is provided on an outer circumferential portion of the film member.
